(19) United States
(12) Patent Application Publication
(10) Pub. No.: US 2011/0112598 A1
(43) Pub. Date: May 12, 2011

(54) Biventricular Cardiac Stimulator

(75) Inventors: Michael Vollkron, Pressbaum (AT); Thomas Kraemer, Nuernberg (DE); Michael Lippert, Ansbach (DE); Gerald Czygan, Buckenhof (DE)

(73) Assignee: BIOTRONIK CRM Patent AG, Baar (CH)

(21) Appl. No.: 12/941,767

(22) Filed: Nov. 8, 2010

Related U.S. Application Data
(60) Provisional application No. 61/259,228, filed on Nov. 9, 2009.

(51) Int. Cl. A61N 1/08; A61N 1/362

(52) U.S. Cl. 607/27

(57) ABSTRACT

A biventricular cardiac stimulator is disclosed, comprising a right ventricular stimulation unit, a left ventricular stimulation unit, and a pacemaker timer. In order to detect the effect of a particular atrioventricular delay time (AVD) and a particular interventricular delay time (VVD), the cardiac stimulator has a detector for sensing a hemodynamic benefit. To optimize AVD and VVD, the pacemaker timer is connected to a memory for a particular instantaneous value for the atrioventricular delay time (AVDᵢₙₛₜ) and the interventricular delay time (VVDᵢₙₛₜ), and may be used to trigger at certain points in time at least one right ventricular trigger signal and one left ventricular trigger signal, based on new values for the atrioventricular delay time (AVDₜₑₛₜ) and the interventricular delay time (VVDₜₑₛₜ) which differ from the instantaneous values for the atrioventricular delay time (AVDᵢₙₛₜ) and the interventricular delay time (VVDᵢₙₛₜ).

### RELATED APPLICATION

This patent application claims the benefit of U.S. Provisional Patent Application No. 61/259,228, filed on Nov. 9, 2009, which is hereby incorporated by reference in its entirety.

### TECHNICAL FIELD

The present disclosure relates to a biventricular cardiac stimulator comprising a right ventricular sensing unit and a left ventricular sensing unit, and a pacemaker timer which, among other things, determines the times for emitting stimulation pulses, for example right ventricular or left ventricular stimulation pulses. The right ventricular sensing unit may be connected to a right ventricular sensing electrode, whereas the left ventricular sensing unit may be correspondingly connected to a left ventricular sensing electrode.

### BACKGROUND

Biventricular cardiac pacemakers are typically used for stimulating the right and the left ventricles of a heart, for example to perform cardiac resynchronization therapy (CRT). For this purpose a biventricular cardiac stimulator typically has a right ventricular stimulation unit and a left ventricular stimulation unit, each of which may be connected to at least one right ventricular or one left ventricular stimulation electrode, respectively.

The right ventricular sensing electrode(s) and the right ventricular stimulation electrode(s) are typically attached to a right ventricular electrode line, whereas the left ventricular sensing electrode(s) and the left ventricular stimulation electrode(s) are components of a left ventricular electrode line. Such left ventricular electrode lines are typically provided to be pushed through the coronary sinus until reaching the vicinity of the left ventricle, and are therefore also referred to as CS electrode lines.

A pacemaker timer determines the times at which stimulation pulses are to be sent to the respective cardiac chambers on the basis of stimulated or sensed events. A stimulated event is the emission of a stimulation pulse which results in a contraction of the corresponding cardiac chamber. A sensed event, also referred to as a natural or intrinsic event, is an autonomous contraction of the cardiac chamber, which is detected via a corresponding sensing electrode. The corresponding sensing electrode detects the electric potentials which accompany a natural contraction of a corresponding cardiac chamber. These electric potentials are amplified and are evaluated by the pacemaker control system, in particular the pacemaker timer.

Existing cardiac devices are known to determine times for emitting a particular stimulation pulse, but to suppress (inhibit) the emission if an autonomous contraction of the respective cardiac chamber, i.e., an intrinsic event in this cardiac chamber, is detected within a given interval before the provided stimulation time. When the cardiac stimulator has such a design, it stimulates the respective cardiac chamber only when needed, and therefore the corresponding operating mode is also referred to as “demand mode.”

As stated above, the times at which a particular next stimulation pulse is provided for a respective chamber are determined by the pacemaker timer on the basis of sensed or stimulated events. For this purpose the pacemaker timer is designed in a known manner so that the chambers of a heart contract in a time sequence which resembles as closely as possible the manner in which a healthy heart autonomously contracts as a function of the particular hemodynamic demand. For example, a contraction of the right atrium, after an atrioventricular conduction period, is followed by a contraction of the right ventricle. The pacemaker timer analogously determines the time for emitting a next right ventricular stimulation pulse on the basis of an atrioventricular delay time (AV delay, or AVD) which is triggered by a stimulated or detected event in the right atrium. In order to specify the AVD it is necessary to identify the time of atrial activation or contraction. In the simplest case this may be performed by detection or stimulation via an atrial electrode. Alternatively, this time may be determined using the following methods, for example: Analysis of IEGM signals measured between ventricular electrodes and the housing (far-field); analysis of signals from pressure sensors, volume sensors, or flow sensors, for example in the right atrium (RA) or left atrium (LA); or analysis of signals from acceleration sensors in or on the RA or LA.

The atrioventricular delay time is advantageously variable in such a way that the pacemaker timer may be optimally adapted to a particular hemodynamic demand and to the individual needs of a particular patient.

For a cardiac pacemaker which stimulates both the right atrium and the right ventricle, the pacemaker timer also determines the time of a next atrial stimulation after a VA delay time which follows a particular stimulated or detected ventricular event and which depends primarily on a heart rate which is adapted as closely as possible to the hemodynamic demand. A cardiac stimulator which has a pacemaker timer and is able to adapt the stimulation rate to the hemodynamic demand is referred to as a rate-adaptive cardiac stimulator.

For biventricular cardiac stimulators the left ventricle may also be stimulated in order to synchronize the actions (contractions) of the right ventricle and the left ventricle within the scope of cardiac resynchronization therapy (CRT). In this regard, also playing a role for the pacemaker timer is an interventricular delay time (VV delay, or VVD), which describes the time delay between the provided emission of a right ventricular stimulation pulse and the provided emission of a left ventricular stimulation pulse and which may also be zero or negative, so that, for example, emission of a left ventricular stimulation pulse may also be provided before the emission of a right ventricular stimulation pulse. This interventricular delay time also is preferably variable, in the sense that the pacemaker timer may be adjusted to individual needs and current requirements for a particular patient.

Because the basic functional mechanism of biventricular cardiac stimulators, including those which are also able to stimulate the right atrium and therefore are three-chamber stimulators, may be assumed to be known to those skilled in the art, a further detailed description is not provided here.

### SUMMARY

A feature disclosed herein provides an improved cardiac stimulator for cardiac resynchronization therapy. This is achieved by use of a biventricular cardiac stimulator comprising a right ventricular stimulation unit which is or may be connected to a right ventricular stimulation electrode which upon receipt of a right ventricular trigger signal emits at least one stimulation pulse via the right ventricular electrode, and a left ventricular stimulation unit which is or may be connected to a left ventricular stimulation electrode which upon receipt of a left ventricular trigger signal emits at least one stimulation pulse via the left ventricular electrode, and a pacemaker timer which is connected to the right ventricular stimulation unit and to the left ventricular stimulation unit and which may be used to emit right ventricular trigger signals and left ventricular trigger signals at the end of an atrioventricular delay time (AVD) or an interventricular delay time (VVD). In order to detect the effect of a particular atrioventricular delay time (AVD) and a particular interventricular delay time (VVD), the cardiac stimulator has a detector for detecting a hemodynamic benefit (HDB). In the present and following discussions, an HDB refers either to an absolute measured value or to a difference relative to a reference state. For optimization of AVD and VVD the pacemaker timer is connected to a memory for a particular instantaneous value for the atrioventricular delay time (AVDᵢₙₛₜ) and the interventricular delay time (VVDᵢₙₛₜ), and may be used to trigger at certain points in time at least one right ventricular trigger signal and one left ventricular trigger signal, based on test values for the atrioventricular delay time (AVDₜₑₛₜ) and the interventricular delay time (VVDₜₑₛₜ) which differ from the instantaneous values for the atrioventricular delay time (AVDᵢₙₛₜ) and the interventricular delay time (VVDᵢₙₛₜ), and to store the HDB determined for a particular value pair of AVDₜₑₛₜ and VVDₜₑₛₜ and associated with the two values for AVDₜₑₛₜ and VVDₜₑₛₜ in the manner of a matrix. The HDB may also be stored in multiple matrices which are selected based on given states, for example heart rate range, atrial event, activity level, or the like.

The HDB is characterized by one or more values of one or more physiological parameters, for example the value of a parameter which results from the variation over time of the intracardiac impedance. The particular value which describes the HDB may also be a value composed of multiple (sub)values.

Suitable physiological parameters may also be derived from other sensors, such as sensors for pressure, flow, volume, acceleration, heart sounds, velocity profiles, or the like.

The cardiac stimulator disclosed allows simultaneous optimization of the atrioventricular delay time (AVD) and the interventricular delay time (VVD) during continuous operation of a cardiac stimulator for cardiac resynchronization therapy (CRT). To this end, the cardiac stimulator incorporates a tracking mechanism which tracks both parameters so rapidly that they are able to trace the typical changes in load, heart rate, bodily position, etc.

The pacemaker timer preferably evaluates stored values for possible atrioventricular delay times and interventricular delay times and the associated HDB for determining suitable test values for the atrioventricular delay time (AVDₜₑₛₜ) and the interventricular delay time (VVDₜₑₛₜ). In this regard, the pacemaker timer is particularly preferably used to select test values for the atrioventricular delay time and the interventricular delay time for which no value has yet been stored which characterizes the HDB associated with these values for the atrioventricular delay time and the interventricular delay time.

Alternatively, the pacemaker timer may be used to investigate the currently active matrix, or also matrices outside the current state, for earlier results which promise an improvement over the currently achieved HDB, and to reinvestigate these results, and optionally also the parameters thereof, for test purposes.

Alternatively or additionally, the pacemaker timer may be used to select test values for the atrioventricular delay time and the interventricular delay time for which a particular associated value which characterizes the HDB was determined at a point in time that precedes a specified time.

The pacemaker timer is preferably used to select test values for the atrioventricular delay time and the interventricular delay time for which the highest HDB is stored. This preferably occurs when there are no more empty or outdated matrix fields.

As a result of these features of the pacemaker timer, after the shortest possible time the maximum number of all suitable values for the atrioventricular delay time and for the interventricular delay time are stored which characterize the HDB associated with the particular values for the atrioventricular delay time and the interventricular delay time. On account of the latter-referenced variant, the particular stored values which characterize the HDB are the most current values possible.

According to a further variant, the pacemaker timer may be used to determine test values based on an operating point defined by current values for the atrioventricular delay time (AVDᵢₙₛₜ) and the interventricular delay time (VVDᵢₙₛₜ) situated in a measurement direction starting from the operating point, the pacemaker timer being further designated to determine the measurement direction by evaluating stored values for the atrioventricular and the interventricular delay times as well as associated values which characterize the particular HDB. This allows the pacemaker timer to preferably select test values which result in a higher expected HDB based on previously recorded values for the HDB. The pacemaker timer is thus able to find the maxima of the HDB in the shortest possible time. To allow determination of the optimal direction, also for small changes which are not amenable to direct evaluation, trend analysis may also be used over several results determined in immediate temporal and spatial proximity.

In any case, a pacemaker timer is preferred which always uses the test values for the atrioventricular delay time and the interventricular delay time to produce new operating values for the atrioventricular delay time and the interventricular delay time for further normal stimulation when an HDB associated with the particular currently tested test values for the atrioventricular delay time (AVDₜₑₛₜ) and the interventricular delay time (VVDₜₑₛₜ) is greater than for the previous operating values. A check is also preferably made to determine whether the difference in the HDB exceeds a specified minimum value.

According to a further preferred design variant, the pacemaker timer may be used to reject test values for the atrioventricular delay time and the interventricular delay time when these test values or the value associated therewith which characterizes the HDB do not satisfy the specified quality conditions. The pacemaker timer is thus able to exclude unsuitable values for the atrioventricular or the interventricular delay time. If individual patient-related limitations regarding the reasonable and definite range of the AVD/VVD parameters are known, it is also possible to limit the AVD/VVD range.

If the quality conditions for at least one specified time or number of cycles are not satisfied, the pacemaker timer is preferably designed to automatically switch to an alternative method for determining AVD and VVD, for example, a method which reduces noise by long-term averaging, or even a method which determines AVD and VVD using a permanent programmed parameter as a function of HR and the type of atrial event.

According to further preferred design variants, the pacemaker timer may be used to permit intrinsic atrioventricular conduction, and to facilitate same by corresponding variations in the operating values for AVD and VVD.

Furthermore, the pacemaker timer is preferably used to select and use randomly determined values for AVDₜₑₛₜ and VVDₜₑₛₜ in repeating time intervals, independently of an instantaneous measurement direction MR, in order to prevent the pacemaker timer from continually adhering to stimulation parameters, in particular AVD and VVD values, which result only in a local, not a global, maximum of the HDB.

Further advantages of the invention result from the following description of one exemplary embodiment.

### DESCRIPTION OF THE DRAWINGS

The invention is explained in greater detail based on one exemplary embodiment, with reference to the Figures, which show the following:

FIG. 1: shows a device according to a preferred embodiment in the form of a cardiac pacemaker, together with electrodes attached thereto. The location of the electrodes in relation to a human heart is diagrammatically illustrated; the lower left part shows a diagrammatic illustration of the variation over time of the current supplied for impedance measurement;

FIG. 2: shows a block diagram of essential components of the cardiac pacemaker shown in FIG. 1;

FIG. 3: shows a block diagram of essential components of the cardiac pacemaker shown in FIG. 1 in a variant as an alternative to FIG. 2;

FIG. 4: shows a three-dimensional contour diagram by way of example which represents an HDB for various values of AVD and VVD.

FIG. 5: shows a general flow diagram for describing the principle of operation of the pacemaker control system and the pacemaker timer thereof for the case of testing values of AVD and VVD and storage of the particular test results;

FIG. 6: shows a flow diagram by way of example for describing the principle of operation of the pacemaker control system and the pacemaker timer thereof for the case of testing values for the atrioventricular delay time AVD;

FIG. 7: shows a flow diagram by way of example for describing the principle of operation of the pacemaker control system and the pacemaker timer thereof for the case of testing values for the interventricular delay time VVD;

FIG. 8: shows a flow diagram by way of example for describing the principle of operation of the pacemaker control system and the pacemaker timer thereof for determining test values for AVD and VVD and storage of the measurement results for the particular associated HDB; and

FIG. 9: shows an alternative flow diagram for illustrating the principle of operation of the pacemaker control system and the pacemaker timer thereof for the case of testing values for AVD and VVD and storage of the particular test results.

### DETAILED DESCRIPTION

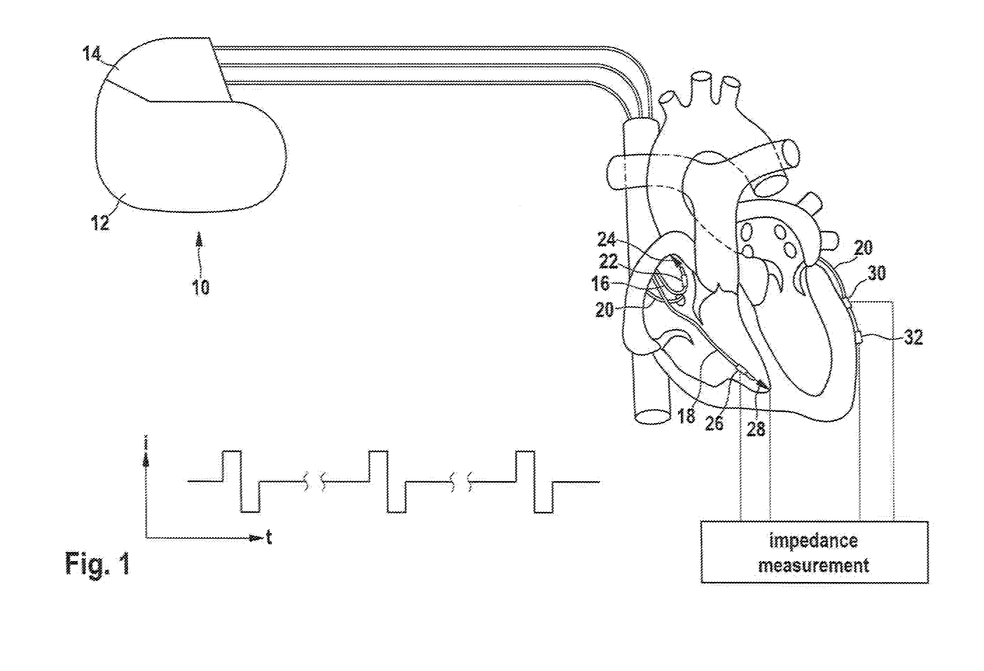

FIG. 1 shows a biventricular cardiac stimulator 10 which is connected in the right atrium, in the right ventricle, and near the left ventricle of a heart via electrode lines having sensing and stimulation electrodes.

The cardiac stimulator 10 preferably has a hermetically sealed metal housing 12 and a connecting head (header) 14 made of substantially transparent plastic and having multiple sockets for connecting the electrode lines. The electrode line terminals in the header 14 are electrically connected to a control circuit inside the housing for the cardiac pacemaker 10.

A total of three electrode lines are connected to the electrode line terminals, namely, a right atrial electrode line 16, a right ventricular electrode line 18, and a left ventricular electrode line 20. The right atrial electrode line 16 bears a right atrial ring electrode 22 and a right atrial tip electrode 24. The right ventricular electrode line 18 bears a right ventricular ring electrode 26 and a right ventricular tip electrode 28. The left ventricular electrode line 20 is guided through the right atrium of the heart schematically illustrated in FIG. 1 and the coronary sinus of the heart until reaching the periphery of the left ventricle. The left ventricular electrode line 20 bears a left ventricular ring electrode 30 and a left ventricular tip electrode 32.

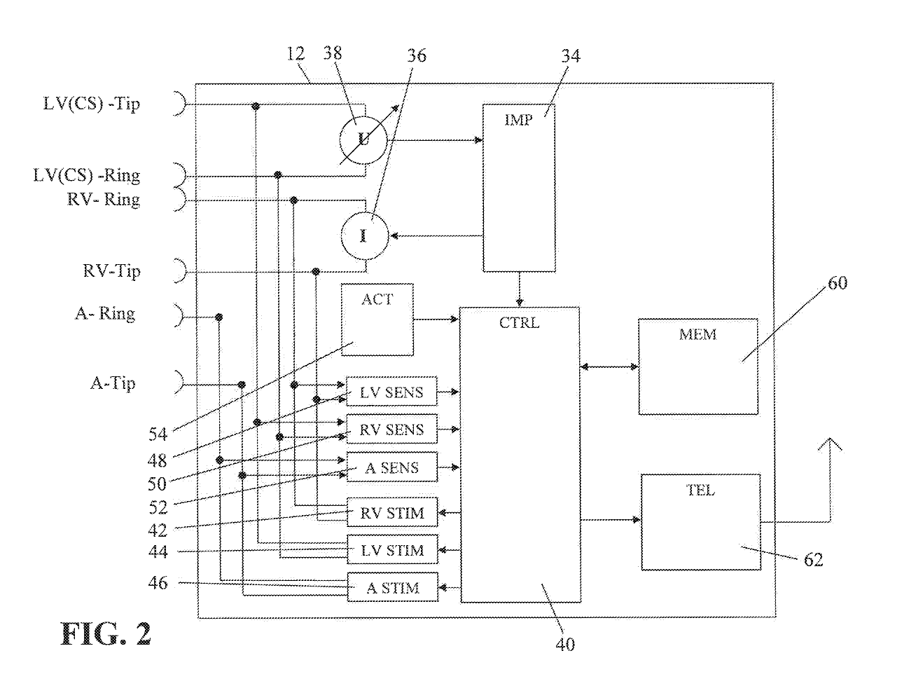

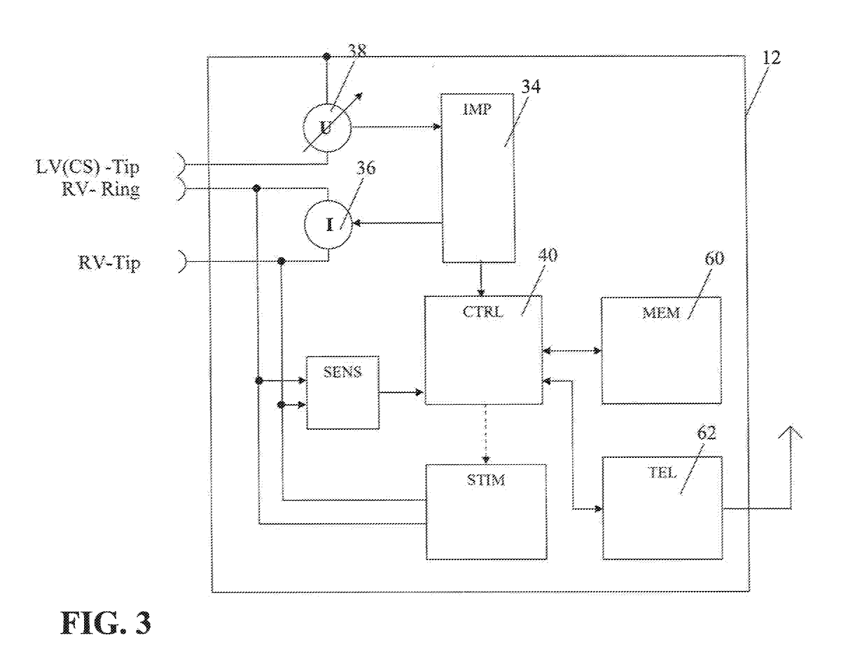

For determination of an HDB, the cardiac stimulator 10 has an impedance measuring unit (see FIGS. 2 and 3) which, in the variant illustrated in FIG. 2, is connected to the right ventricular ring electrode 26 and the right ventricular tip electrode 28 as well as to the left ventricular ring electrode 30 and the left ventricular tip electrode 32 for purposes of impedance measurement.

A biphasic pulsed measuring current as diagrammatically illustrated in the lower left part of FIG. 1 is supplied via the right ventricular ring electrode 26 and the right ventricular tip electrode 28, i.e., in the right ventricle. The voltage produced by the current is measured via the left ventricular ring electrode 30 and the left ventricular tip electrode 32. The roles of the electrodes for current feed and voltage measurement may also be reversed. As shown in the lower left part of FIG. 1, the current for the impedance measurement is supplied in biphasic pulses, with two antiphase constant current pulses following one another in immediate succession and in each case forming a pulse packet. The individual pulse packets are spaced at time intervals which are much greater than the duration of the particular pulse packet. The two direct current pulses within the pulse packet each have the same amplitude but different polarity, and have the same length. Typical values for the maximum current of the direct current pulses are between 50 and 600 microamperes. A typical pulse duration of an individual current pulse may be 15 microseconds. The pulse packets are generated at regular intervals, typically with repetition frequencies between 30 and 300 Hz.

In a departure from the illustration in the lower left part of FIG. 1, the two direct current pulses of a pulse packet may also follow one another at time intervals which correspond to the duration of one direct current pulse. This results in a gap in the duration of a direct current pulse between two antiphase direct current pulses of a pulse packet, during which no direct current is supplied. In a further variant the pulse packets are emitted in alternating phases; i.e., in strict alternation a pulse packet starts with a negative direct current pulse, for example, and ends with a positive direct current pulse, and the subsequent pulse packet starts with a positive direct current pulse and ends with a negative direct current pulse, and so forth. This alternating-phase emission of pulse packets prevents charging of the electrode boundary surfaces and avoids artifacts.

FIGS. 2 and 3 each show block diagrams presenting essential components of a circuit inside the housing 12 of the cardiac stimulator 10. These components include an impedance measuring unit 34, which on the one hand is connected to a constant current generator 36 which generates a pulsed biphasic constant current and which emits stimulation pulses via a terminal RV-Ring for the right ventricular ring electrode 26 and a terminal RV-Tip, for the right ventricular tip electrode 28. The impedance measuring unit 34 is also connected to a voltage measurement unit 38 which is connected to two terminals via which the particular voltage that produces the constant current supplied for impedance measurement purposes is detected. In the preferred variant according to FIG. 2, the voltage measurement unit 38 is connected to a terminal LV-Ring for the left ventricular ring electrode 30 and to a terminal LV-Tip for the left ventricular tip electrode 32. In an alternative variant (see FIG. 3) the voltage measurement unit 38 is connected to a terminal LV-Tip for the left ventricular tip electrode 32 and also to the electrically conductive housing 12 for the cardiac pacemaker 10.

The housing 12 for the cardiac stimulator 10 also contains a pacemaker control system 40 which includes a pacemaker timer (not illustrated). The pacemaker control system 40 is connected to a right ventricular stimulation unit 42, a left ventricular stimulation unit 44, and an atrial stimulation unit 46. The right ventricular stimulation unit 42 is connected to an electrical terminal for the right ventricular ring electrode RV-Ring and to an electrical terminal for the right ventricular tip electrode RV-Tip. Correspondingly, the left ventricular stimulation unit 44 is connected to an electrical terminal for the left ventricular ring electrode RV-Ring and to an electrical terminal for the left ventricular tip electrode RV-Tip. The atrial stimulation unit 46 is analogously connected to an electrical terminal for the atrial ring electrode A-Ring and to an additional electrical terminal for the atrial tip electrode A-Tip.

The stimulation units 42, 44, and 46 are each designed to generate a stimulation pulse on the basis of a trigger signal from the pacemaker control system 40, and to emit this stimulation pulse via the electrical terminals to the electrodes connected thereto and, in the implanted state, to the cardiac tissue of the respective cardiac chamber. The stimulation pulse is dimensioned so that it results in a stimulated contraction of the cardiac tissue, as known to one skilled in the art in the field of cardiac rhythm management.

The pacemaker control system 40 is also connected to a left ventricular sensing unit LV-Sense 48, a right ventricular sensing unit RV-Sense 50, and an atrial sensing unit A-Sense 52. As for the corresponding stimulation units, the sensing units 48, 50, and 52 are also connected to the electrical terminals for the corresponding electrodes. The sensing units 48, 50, and 52 are used in a known manner to amplify the electrical potentials recorded at the attached electrodes and to evaluate the resulting signal for signal characteristics which indicate a natural contraction of the respective cardiac chamber. In the simplest case, the particular sensing unit 48, 50, or 52 detects exceedance of a threshold value by the particular signal to be evaluated, and thereupon generates a sensing signal which characterizes a sensed event which is evaluated as an indication of a natural contraction of the corresponding cardiac chamber. The respective sensing unit 48, 50, or 52 sends these signals to the pacemaker control system 40. The pacemaker control system 40 responds to a particular sensing signal by, for example, suppressing in a known manner the emission of a next, previously provided stimulation pulse. At the same time, a sensing signal may trigger an interval which determines the time of emission of a next provided stimulation pulse. This is also basically known.

An activity sensor 54 is likewise connected to the pacemaker control system 40, and allows the pacemaker control system 40 or the pacemaker timer thereof to adapt a particular stimulation rate to the hemodynamic demand of a patient; the cardiac stimulator 10 is therefore rate-adaptive. The activity sensor 54 may be, for example, an accelerometer, an impedance sensor (for example, a minute volume impedance sensor or “closed-loop” impedance sensor), or another physiological sensor for detecting an instantaneous load on the patient.

As previously mentioned, an essential function of the pacemaker timer, and thus of the pacemaker control system 40, is to determine suitable times for emitting a particular stimulation pulse to the respective chamber of a heart in such a way that the stimulation of the heart results in the greatest possible HDB. In addition to the function of determining a general stimulation rate (the rate at which identical heart actions, for example ventricular contractions, follow one another) which has maximum dependence on the bodily activity of the patient, the pacemaker timer has the function of determining suitable intervals which within a cardiac cycle define the times of the stimulation pulse emission to the individual chambers. These intervals are in particular an atrioventricular delay interval AVD which is started with a natural (sensed) atrial event or an atrial stimulus, and at its end a right ventricular stimulation pulse is triggered, provided that it is not suppressed as the result of a previously detected, natural right ventricular event, and an interventricular delay interval VVD which determines the time interval between a provided right ventricular stimulus and a provided left ventricular stimulus. Typically, the time of the provided emission of a left ventricular stimulus is calculated starting at the point in time when a right ventricular stimulus is provided. The interventricular delay time VVD may be positive as well as negative; i.e., the provided left ventricular stimulus may be present before or after the provided right ventricular stimulus of a time cycle.

The cardiac stimulator 10 is used to determine as rapidly as possible suitable values for the atrioventricular delay time AVD and the interventricular delay time VVD which are adapted to a particular situation and a particular patient. For this purpose, at regularly or irregularly repeating times the pacemaker control system 40 tests each pair of values for the atrioventricular delay time AVD and the interventricular delay time VVD. The HDB associated with these respective pairs of values AVDₜₑₛₜ and VVDₜₑₛₜ to be tested for the atrioventricular delay time or the interventricular delay time is determined by the pacemaker control system 40 with the assistance of the impedance measuring unit 34. The variation over time of the intracardiac impedance, determined using the impedance measuring unit 34, is an indication of a particular achieved HDB. Alternatively, this may be determined by evaluating a pressure-volume diagram, as described in detail in a related German Patent Application No. DE 10 2009 002 397.6.

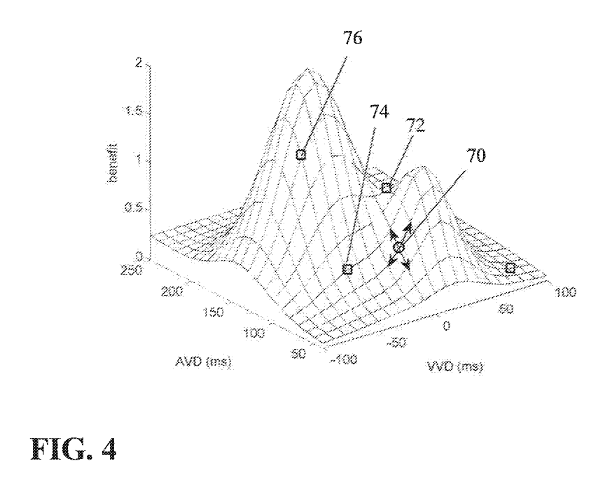

The HDB determined for a particular value pair of AVDₜₑₛₜ and VVDₜₑₛₜ is associated with the two values for AVDₜₑₛₜ and VVDₜₑₛₜ and stored in the manner of a matrix. To this end, the pacemaker control system 40 is connected to a memory 60. The memory 60 allows the pacemaker control system 40 and in particular the pacemaker timer to access these values at a later time in order to use the most optimum values for AVD and VVD for the time control for a particular instantaneous demand. One example of a values matrix plotted in this manner is illustrated in FIG. 4. In FIG. 4, the HDB for combinations of AVD and VVD values is plotted in a schematic contour diagram. It is useful to create several such contour diagrams as a function of heart rate, type of atrial event, and activity level, because the corresponding optimum values tend to shift according to the applied load.

FIG. 4 shows by way of example an instantaneous setting for the atrioventricular delay time AVDᵢₙₛₜ and the interventricular delay time VVDᵢₙₛₜ, with AVDᵢₙₛₜ=80 ms and VVDᵢₙₛₜ=−10 ms. This operating point 70 is illustrated by a circle in FIG. 4, accompanied by arrows that denote various changes in direction for AVD and VVD. FIG. 4 shows, also by way of example, that the area representing the HDB may have three maxima, namely, a first local maximum 72 at AVD=110 ms and VVD=0 ms and a local maximum 74 at AVD=90 ms and VVD=−50, as well as a global maximum 76 at AVD=130 ms and VVD=−60 ms. In order to record values which may be represented in the form of a matrix corresponding to the points illustrated in FIG. 4, the pacemaker is used to vary the atrioventricular delay time and the interventricular delay time at specified times T_{VAR}, and to use test values for these delay times AVDₜₑₛₜ and VVDₜₑₛₜ which differ from the instantaneous values of these delay times AVDᵢₙₛₜ and VVDᵢₙₛₜ. These test values for the atrioventricular delay time AVDₜₑₛₜ and the interventricular delay time VVDₜₑₛₜ result in an HDB which, as previously described, is quantified by impedance measurement or by recording and evaluation of a pressure-volume diagram, or by use of some other hemodynamic sensor. The value thus obtained which characterizes the particular HDB is stored in association with the particular value pair AVDₜₑₛₜ and VVDₜₑₛₜ used.

In the selection of the particular values to be tested for the atrioventricular delay time and the interventricular delay time, after a specified number of test measurement cycles the pacemaker timer queries the previously stored values in order to check whether suitable value pairs of AVD and VVD are still available which have not been previously tested and for which therefore no value is stored which characterizes the HDB. In this case the pacemaker timer tests operating points which have not been previously tested when there are no other entries in the matrix which allow a global optimum value to be presumed which is outside the immediate vicinity of the operating point.

According to one preferred variant, for each value triplet AVD, VVD, and HDB, the date the particular value triplet was formed is also stored. Before testing further values of AVD and VVD, the pacemaker timer queries the memory 60 regarding the age of the previously tested value pairs, and preferably tests value pairs which are relatively old.

The pacemaker timer is also designed to select such test values for the atrioventricular delay time and the interventricular delay time for which the highest HDB is stored. This preferably occurs when there are substantially no more empty or outdated matrix fields.

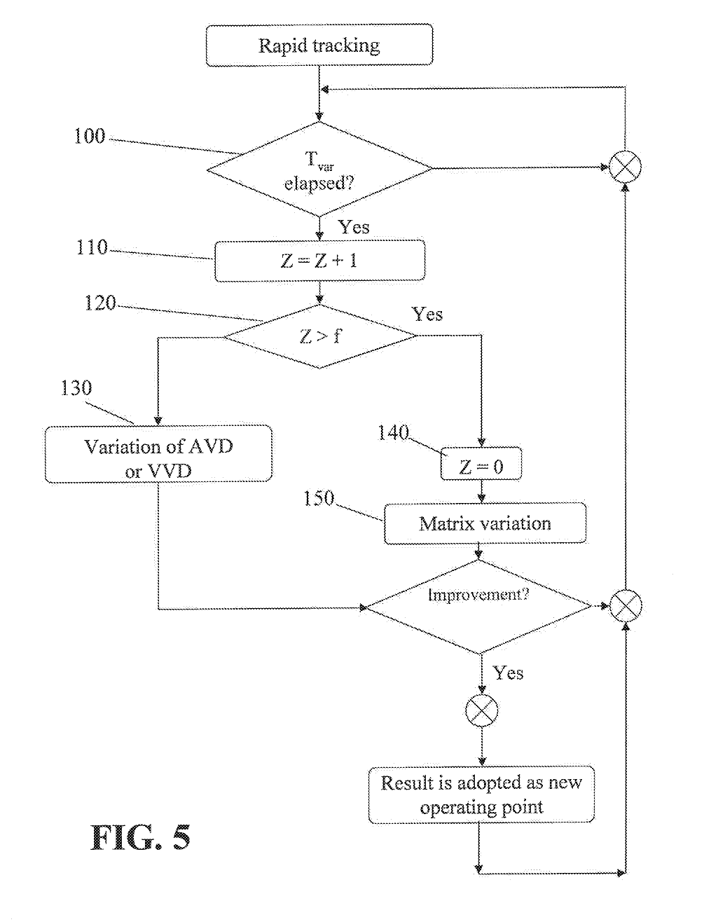

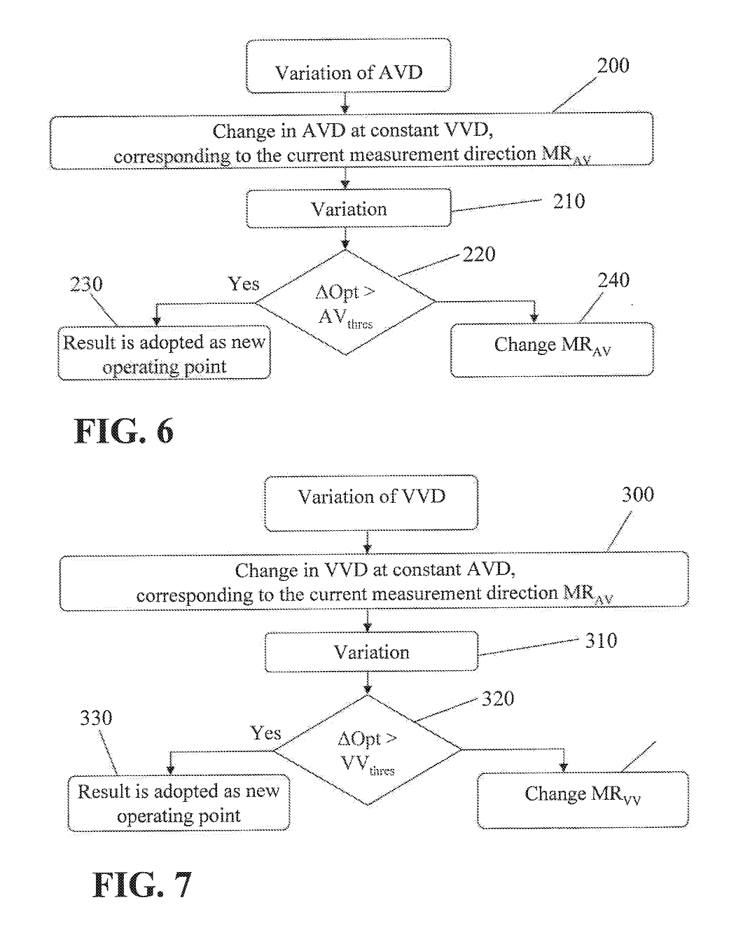

This targeted procedure of the pacemaker timer is illustrated in greater detail in the flow diagrams in FIGS. 5 through 9. FIG. 5 shows that variation of the values for AVD and VVD is repeated in time intervals of T_{VAR} (100). If the particular time interval T_{VAR} has elapsed, a counter Z is incremented (110) and AVD and VVD (130) may be varied over a number of f cardiac cycles (120). A procedure for variation of the atrioventricular delay time is illustrated in FIG. 6, and a procedure for variation of the interventricular delay time is illustrated in FIG. 7.

For the particular variation, the AVD or VVD changes with respect to a particular instantaneous operating point in the direction of an instantaneous measurement direction MR_{AV} or MR_{VV} (see steps 200 and 300, respectively). The pacemaker timer uses the delay times AVDₜₑₛₜ and VVDₜₑₛₜ to be tested; (see FIG. 6, step 210 and FIG. 7, step 310). In both cases, after use of the values to be tested, the atrioventricular delay time AVDₜₑₛₜ and the interventricular delay time VVDₜₑₛₜ are checked to determine whether a change in HDB, ΔOpt (the difference in the hemodynamic state between the test values AVDₜₑₛₜ and VVDₜₑₛₜ and the previous operating values for AVDₜₑₛₜ and VVDₜₑₛₜ), is greater than a particular threshold value AVₜₕᵣₑₛ or VVₜₕᵣₑₛ for hemodynamic improvement as a result of varying AVD or VVD (steps 220 and 320, respectively). The currently tested pair of AVD and VVD values is not adopted as a new instantaneous operating point having the values AVD_{new}=AVDₜₑₛₜ and VVD_{new}=VVDₜₑₛₜ unless the change in the optimization criterion ΔOpt is greater than the particular specified threshold value AVₜₕᵣₑₛ or VVₜₕᵣₑₛ; (see steps 230 and 330, respectively.) If the value ΔOpt which characterizes the change in the optimization criterion does not exceed the particular threshold value AVₜₕᵣₑₛ or VVₜₕᵣₑₛ, the pacemaker timer changes the measurement direction MR_{AV} or MR_{VV} in order to possibly find better suitable test values AVDₜₑₛₜ and VVDₜₑₛₜ and therefore possibly new operating values AVD_{new} and VVD_{new} (see steps 240 and 340, respectively).

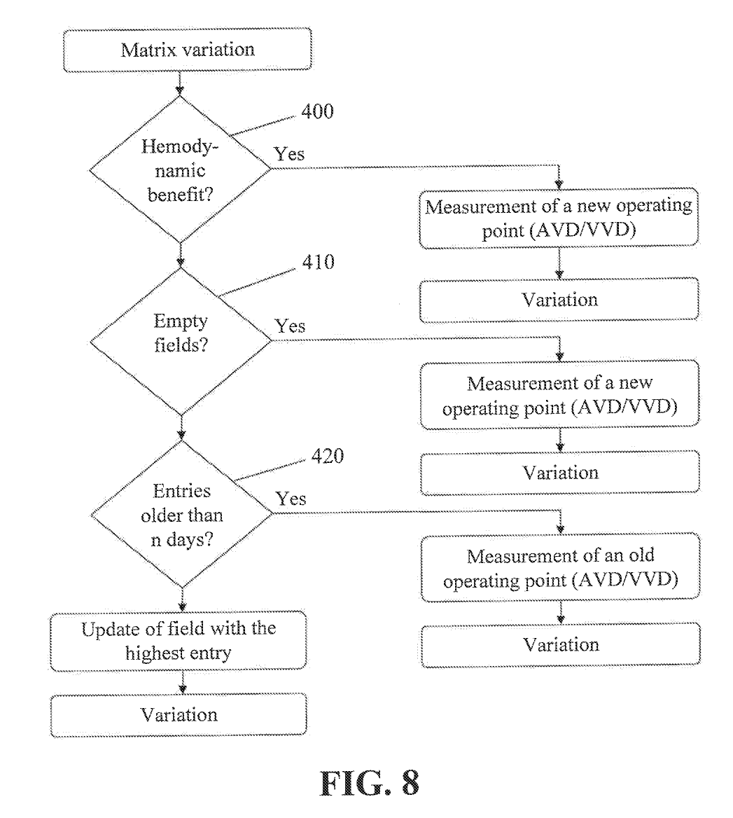

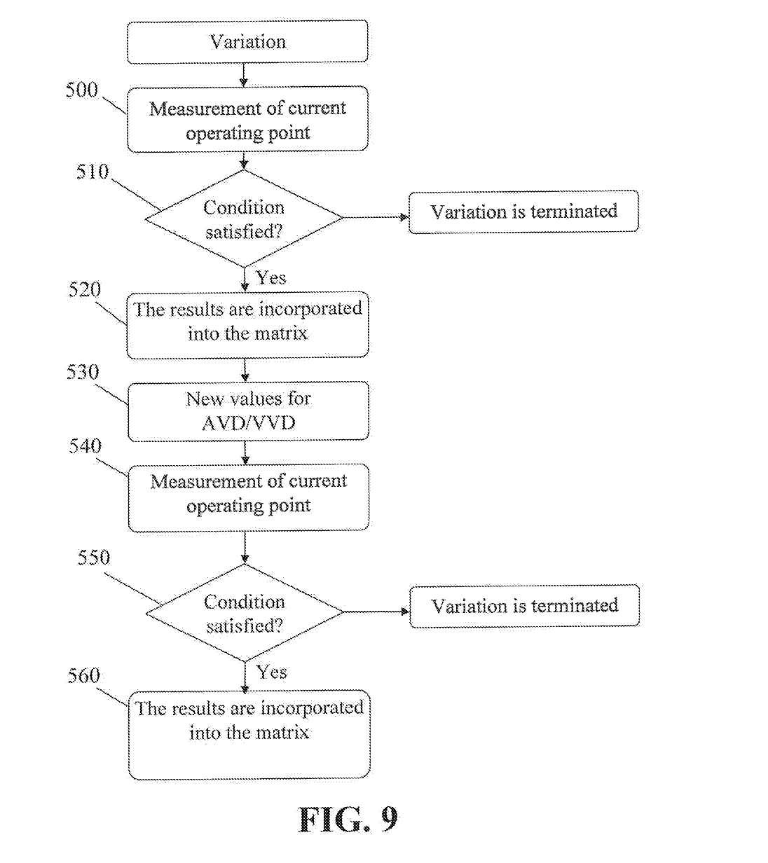

When the pacemaker timer has varied the atrioventricular delay time and the interventricular delay time in this manner, and has tested the number of values of the atrioventricular delay time and the interventricular delay time to be tested which are defined by the counter Z and has optionally found a new operating point, the pacemaker timer resets the counter Z to 0 (140) and performs a matrix variation (150) as shown in FIG. 8. As illustrated in FIG. 8, the pacemaker timer preferably selects values for AVDₜₑₛₜ and VVDₜₑₛₜ for which no value that describes the HDB has been detected (see 400). If substantially all possible operating points have already been measured, the pacemaker timer checks whether some operating points were measured more than n days in the past (410), and then tests the values for AVD and VVD associated with the particular operating point. If an HDB is present in the matrix which promises better results than is the case for the instantaneous operating point, this value pair is remeasured and optionally adopted as the new operating point. A prerequisite, however, is that this AVD/VVD combination is not in the immediate vicinity of the instantaneous operating point (420).

The pacemaker timer takes into account specified quality and termination criteria when testing various operating points, as illustrated in FIG. 9. A current operating point (500) is measured instantaneously by recording a hemodynamically relevant sensor signal (the impedance, for example) for a sufficient number of successive cardiac cycles in order to ensure adequate noise suppression. A check is made as to whether the hemodynamic signal, for example the impedance, determined over 2″ cardiac cycles satisfies specified quality conditions (510). If this is not the case, the test is terminated with test values AVDₜₑₛₜ and VVDₜₑₛₜ. The results are stored as a value triplet; i.e., the particular sensor output signal (the impedance, for example) is incorporated into the matrix defined by AVD and VVD (520) only when the quality conditions are satisfied. New values for AVD and VVD are then tested (530). These values form a new instantaneous operating point, which is precisely measured (540) as described above. In this case as well, satisfaction of the quality conditions (550) is retested. Only when the quality conditions are satisfied are the values for the tested delay times AVDₜₑₛₜ and VVDₜₑₛₜ also stored, i.e., incorporated into the matrix (560). Otherwise the test is terminated. The quality conditions used in steps 510 and 550 may be defined by certain frequency criteria or a specified contraction dynamic, or by the noise or interference for the sensor signal.

The pacemaker timer may also be designed to determine the measurement noise from the sensor or sensors used for determining the HDB, and may be further used for parameter optimization. Thus, for example, for favorable noise ratios a quicker reaction time is possible as the result of a lower number of averaged cycles, or the noise sensitivity is reduced by greater increments or longer averages. It is also practical to switch to a different optimization method, depending on the noise.

Thus, the pacemaker timer is used to continuously adapt a value for AVD and VVD to changing conditions, with a response time of 1-10 minutes. Such an adaptation procedure is carried out as follows:

Assuming an instantaneous setting of the atrioventricular delay time at a value AVDᵢₙₛₜ and of the interventricular delay time at a value VVDᵢₙₛₜ, for several heartbeats the pacemaker timer sets “adjacent” values as values AVDₜₑₛₜ and VVDₜₑₛₜ to be tested (test values).

Such “adjacent values” may be the immediate higher and lower values in a predefined list of possible AVD or VVD values, or may be values at a specified interval above and below the currently set values AVDᵢₙₛₜ and VVDᵢₙₛₜ, or may be systematically selected values which achieved particularly good hemodynamic results in earlier measurements, or randomly selected values from all settable values in a given specified interval about the instantaneous values for AVDᵢₙₛₜ and VVDᵢₙₛₜ.

The hemodynamic change ΔOpt for the sought adjacent values AVDₜₑₛₜ and VVDₜₑₛₜ is evaluated on the basis of parameters, derived from one or more sensor signals, for detecting volumetric variables (for example, from the quadrupolar intracardiac impedance measurement, for example with the power supply on the left or right side of the heart), blood flow variables, blood pressure variables, impedance variables, electrical variables, or intervals, accelerations, or velocities. The value of one or more of these parameters represents the particular value characterizing the HDB.

The measurements for determining the values for the particular parameters are performed either once or repeatedly, and average values may then be calculated.

If the measurement result of the parameter characterizing the HDB for adjacent test values AVDₜₑₛₜ and VVDₜₑₛₜ is better for instantaneous operating values AVDᵢₙₛₜ and VVDᵢₙₛₜ, the test values are adopted as new operating values AVD_{new} and VVD_{new}, and a next pair AVDₜₑₛₜ and VVDₜₑₛₜ to be tested is selected in the same measurement direction MR in which the previously tested test values AVDₜₑₛₜ and VVDₜₑₛₜ were situated in relation to the starting values AVDᵢₙₛₜ and VVDᵢₙₛₜ.

If during an optimization step, i.e., a test of a particular AVDₜₑₛₜ and VVDₜₑₛₜ for stability conditions such as frequency or contraction dynamics, a stability criterion is not satisfied, for example if a departure is made from a specified interval about the values at the start of the optimization step, or if the scattering exceeds a predetermined threshold, the associated measurement result for the value characterizing the HDB may be rejected.

In the exemplary embodiment illustrated, the cardiac stimulator 10 determines the particular HDB difference ΔOpt by determining the difference between two measured values derived from a particular intracardiac impedance measurement for each respective value pair AVDᵢₙₛₜ and VVDᵢₙₛₜ and AVDₜₑₛₜ and VVDₜₑₛₜ. These measured values may be values of parameters such as maximum impedance or average impedance, the ratio of maximum impedance to average impedance, or the difference between maximum impedance and average impedance or between average impedance and minimum impedance, the maximum or minimum slope of the impedance signal, etc. Alternatively, the cardiac stimulator may be designed to derive the values characterizing an HDB from pressure-volume diagrams.

In order to test the test values for AVD and VVD, the pacemaker timer, starting from an operating point AVDᵢₙₛₜ and VVDᵢₙₛₜ, first changes the value of the atrioventricular transition time and then, likewise starting from the operating point, changes the value of the interventricular transition time. By evaluation of the optimization criterion the direction with respect to the optimum may be determined, and a suitable measurement direction MR may be correspondingly determined. Both parameters (AVD and VVD) are jointly changed in the direction of this optimum value. In this manner an independent optimization criterion may be selected for the optimization of AVD and VVD.

If the specified stability conditions are satisfied (for example, the average change in AVD per optimization step is below a specified threshold value), after specified time intervals or in an event-controlled manner, the data for the current operating point are stored. These data include, for example, the atrioventricular delay time AVD, the interventricular delay time VVD, the heart rate HR, the bodily load (which is characterized, for example, by a starting value for the activity sensor 54), time, stability conditions, impedances, electrical signals, or hemodynamic measured values. The number of measured values for each AVD-VVD value pair is permanently set; new measured values result in overwriting of older measured values, or new measured values are sorted into groups, for example according to predefined heart rate ranges.

For determination of suitable test values and in particular for finding and maintaining a global hemodynamic optimum, the pacemaker timer is used to store, for example in a matrix, events from previous tests together with the measurement results. This storage of the previously recorded measured values is also used for the pacemaker timer to check all possible AVD/VVD combinations over time, thereby reliably preventing the pacemaker timer from continually adhering to operating values for AVD and VVD which result (only) in a local, not a global, maximum of the HDB.

To this end, the pacemaker timer is designed not to measure just one particular set of closely adjacent test values for AVD and VVD, but, rather, to measure a selection of AVD/VVD combinations which are “farther away,” which contributes to avoidance of local maxima, for example:
AVD/VVD combinations for which the value characterizing the HDB has not yet been measured, or whose last measurement is the least recent;
AVD/VVD combinations for which a comparable activity level (similar heart rate, identical type of atrial event, similar activity sensor starting value, etc.) has been recorded;
AVD/VVD combinations which result in a maximum hemodynamic evaluation (with or without a matching activity level).

The pacemaker timer may also be used for stochastic selection of AVD/VVD combinations to be tested which are within a specified vicinity in order to preferably supplement missing entries. AVD/VVD combinations to be tested may also be selected using methods based on the evaluation of intracardiac electrograms (IEGM).

In addition, the pacemaker timer may be used to make determinations in specific, optionally adjustable time intervals, or, if an IEGM criterion is satisfied, to determine a new AVD/VVD operating point as described above. From that point the above-referenced optimization may in turn be used.

Alternatively, the pacemaker timer may be used to make determinations in specific, optionally adjustable time intervals, or, if a criterion based on changes of, for example, heart rate, contraction dynamics, activity level, or time of day is satisfied, to determine a new AVD/VVD operating point as described above. From that point the above-referenced optimization may in turn be used.

The pacemaker timer is also used to allow intrinsic atrioventricular conduction, and to facilitate same by appropriate variations of the operating values for AVD and VVD. Intrinsic atrioventricular conduction is conduction which results in a natural, nonstimulated contraction of the left or right ventricle, or both ventricles, due to natural stimulus conduction in the heart. The pacemaker timer checks to determine whether the hemodynamic signal or signals satisfy quality criteria. If this is not the case, the pacemaker timer automatically uses an alternative optimization method.

In the event that a hemodynamic measurement of adequate quality is not possible, the pacemaker automatically transfers to normal (nonoptimized) AVD/VVD dynamic parameters.

The functional mechanism of the pacemaker timer may be summarized as follows: Starting from the instantaneous operating point 70 shown in FIG. 4, the adjacent values are measured to approximate the optimum in a stepwise manner (arrows). A randomly selected set of settings (squares) is measured at regular intervals. The same as for the matrix variation, this method is used to “escape” a local maximum (in FIG. 4, for example VVD20, AVD80).

In order to allow reprogramming of the functional mechanism of the pacemaker control system and of the pacemaker timer, or for specifying suitable values for stimulation parameters, the pacemaker control system is connected to telemetry unit 62. By use of such a unit 62, values stored in memory 60, for example measured values such as the particular HDB for various AVD and VVD values, may also be transmitted to a central service center.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of the following claims.

What is claimed is:

1. A biventricular cardiac stimulator comprising:
a right ventricular stimulation unit connected to a right ventricular stimulation electrode which, upon receipt of a right ventricular trigger signal, emits at least one stimulation pulse via the right ventricular electrode;
a left ventricular stimulation unit connected to a left ventricular stimulation electrode which upon receipt of a left ventricular trigger signal emits at least one stimulation pulse via the left ventricular electrode;
a pacemaker timer which is connected to the right ventricular stimulation unit and to the left ventricular stimulation unit and which is used to emit right ventricular trigger signals and left ventricular trigger signals at the end of an atrioventricular delay time (AVD) or an interventricular delay time (VVD); and
a detector for a hemodynamic benefit (HDB), characterized in that the pacemaker timer is connected to a memory for a particular instantaneous value for the atrioventricular delay time (AVDᵢₙₛₜ) and the interventricular delay time (VVDᵢₙₛₜ), and is used to trigger at certain points in time at least one right ventricular trigger signal and one left ventricular trigger signal, based on test values for the atrioventricular delay time (AVDₜₑₛₜ) and the interventricular delay time (VVDₜₑₛₜ) which differ from the instantaneous values for the atrioventricular delay time (AVDᵢₙₛₜ) and the interventricular delay time (VVDᵢₙₛₜ), and to store the HDB determined for a particular value pair of AVDₜₑₛₜ and VVDₜₑₛₜ and associated with the two values for AVDₜₑₛₜ and VVDₜₑₛₜ in the manner of a matrix.

2. The biventricular cardiac stimulator according to claim 1, characterized in that the pacemaker timer is used to evaluate stored values for possible atrioventricular delay times and interventricular delay times together with the associated value characterizing the HDB for determining suitable test values for the atrioventricular delay time (AVDₜₑₛₜ) and the interventricular delay time (VVDₜₑₛₜ).

3. The biventricular cardiac stimulator according to claim 2, characterized in that the pacemaker timer in this regard is used to select test values for the atrioventricular delay time and the interventricular delay time for which no value characterizing the HDB has yet been stored.

4. The biventricular cardiac stimulator according to claim 2, characterized in that the pacemaker timer is used to select test values for the atrioventricular delay time and the interventricular delay time for which a particular associated value which characterizes the HDB was determined at a point in time that precedes a specified time.

5. The biventricular cardiac stimulator according to claim 2, characterized in that the pacemaker timer is used to select test values for the atrioventricular delay time and the interventricular delay time for which an HDB is stored which is better than the current value and is not present in the direct vicinity thereof.

6. The biventricular cardiac stimulator according to claim 1, characterized in that the pacemaker timer is used to determine test values for the atrioventricular delay time and the interventricular delay time which are present in a measurement direction (MR), starting from an operating point defined by instantaneous values of the atrioventricular delay time (AVDᵢₙₛₜ) and the interventricular delay time (VVDᵢₙₛₜ), the pacemaker timer being further designed to determine the measurement direction by evaluating stored values for the atrioventricular and the interventricular delay times as well as associated values which characterize the particular HDB.

7. The biventricular cardiac stimulator according to claim 1, characterized in that the pacemaker timer is used to determine a correct measurement direction (MR) on the basis of trend data of the most recent operating points when the accuracy of the individual measurements is not adequate for this purpose.

8. The biventricular cardiac stimulator according to claim 1, characterized in that the pacemaker timer is designed so that when an improvement in the HDB that is associated with particular currently tested test values for the atrioventricular delay time (AVDₜₑₛₜ) and the interventricular delay time (VVDₜₑₛₜ) exceeds a specified minimum value, the pacemaker timer always uses the test values for the atrioventricular delay time and the interventricular delay time to produce new operating values for the atrioventricular delay time (AVD_{new}) and the interventricular delay time (VVD_{new}) for the further normal stimulation.

9. The biventricular cardiac stimulator according to claim 1, characterized in that the pacemaker timer is used to reject test values for the atrioventricular delay time and the interventricular delay time if these test values or the value associated therewith which characterizes the HDB do not satisfy the specified quality conditions.

10. The biventricular cardiac stimulator according to claim 9, characterized in that the pacemaker timer is used to automatically transfer to default values for AVD and VVD if the quality conditions are not satisfied.

11. The biventricular cardiac stimulator according to claim 1, characterized in that the pacemaker timer is used to allow intrinsic atrioventricular conduction and to facilitate same by corresponding variations in the operating values for AVD and VVD.

12. The biventricular cardiac stimulator according to claim 1, characterized in that the pacemaker timer is used to use randomly determined values for AVDₜₑₛₜ and VVDₜₑₛₜ in repeating time intervals.
